(19) United States
(12) Patent Application Publication
(10) Pub. No.: US 2013/0298338 A1
(43) Pub. Date: Nov. 14, 2013

(54) Skin conditioning method

(71) Applicant: Lina Kennedy, Williamsville, NY (US)

(72) Inventor: Lina Kennedy, Williamsville, NY (US)

(21) Appl. No.: 13/863,097

(22) Filed: Apr. 15, 2013

Related U.S. Application Data
(60) Provisional application No. 61/624,072, filed on Apr. 13, 2012.

(51) Int. Cl. A61K 8/60; A61Q 9/04; G09B 23/28

(52) U.S. Cl. CPC A61K 8/60; G09B 23/28; A61Q 9/04
USPC 8/160; 434/262

(57) ABSTRACT

A skin conditioning program comprises a skin softening and repair step where a natural composition is applied to the skin; a hair extraction and follicle decongestion step where a natural composition is applied to the skin; a follicle detoxification step where a mineral composition is applied to the skin; and skin exfoliation step where a mineral composition is applied to the skin. These program elements are complementary. The skin softening and repair improves the exfoliation; improved exfoliation improves the penetration and efficiency of the follicle detoxification; and the improved detoxification improves paste penetration and sugaring efficiency. The skin conditioning program is designed to establish and to maintain improved skin and hair follicle condition in order to promote more effective hair removal.

### RELATED APPLICATIONS

This US non-provisional patent application is related to U.S. Provisional Patent Application No. 61/624,072 filed by applicant on Apr. 13, 2012 and claims the benefit of that filing date.

### FIELD OF INVENTION

The present invention relates to a method of hair removal and skin conditioning treatment.

### BACKGROUND

Traditionally, hair removal has been performed by waxing. This is typically done in a salon by a body wax professional using hot or cold wax applied to the hair which the clients wish to have removed. The waxing products are made of petroleum based resins and chemicals which can be prone to contamination or cross contamination. With hot waxing, there are risks of burning skin, damaging the dermal cells, scarring the follicle mouth or surrounding area and causing pigmentation. The wax will adhere to the skin cells, or any surface it is applied to, making pulling it away painful. When waxing, you remove the unwanted hair against the natural direction of growth which causes undue levels of pain and becomes the major contributor to ingrown hair. Hair trapped under the skin which can also be painful and unsightly and cause infections.

A hair follicle is a skin organ that produces hair. Hair production occurs in phases, including a growth phase (anagen), and cessation phase (catagen), and a resting phase (telogen). Stem cells are principally responsible for the production of hair.

Anagen is the active growth phase of hair follicles. The roots of the hair are dividing rapidly, adding to the hair shaft. During this phase, the hair grows about 1 cm every 28 days. Scalp hair stays in this active phase of growth for 2-7 years. The amount of time the hair follicle stays in the anagen phase is genetically determined. There are other factors that can affect hair growth and hair loss patterns such as disease, medication, stress, and severe dieting. Generally, genes play the major role. At the end of the anagen phase, the follicle goes into the catagen phase.

### SUMMARY OF INVENTION

“Advanced body sugaring” is a unique and innovative process for the removal of unwanted facial and body hair for men, women and children alike.

In some examples, the methods of the current invention are performed by trained professionals in advanced body sugaring and skin conditioning treatments through salons and spa therapy facilities. In other examples, the methods may be performed at home.

Sugaring requires an entirely different mind-set and approach than for waxing. In the methods described, the all natural sugaring compositions do not adhere to live skin cells, therefore, when the compositions are removed, the composition gently exfoliates dead skin cells and leaves behind freshened skin. There is no after pain, stickiness, bruising or burning.

In one example, the sugaring compositions comprise pure sugar, water and lemon. They are edible and delicious, and since they are water soluble, can easily rinsed away if accidentally applied to an area. The sugaring will extract all hair colors and textures. The sugaring methods remove the hair in the natural direction of the hair growth.

When the sugar paste is applied, it seeps into the hair follicle and helps lubricate that hair root. The sugaring process will successfully treat all skin types and colors while improving ones skin tone and texture. Sugaring will eliminate ingrown hairs, and help prevent new ingrown hairs from developing. Since sugaring is done at body temperature, and requires no heat, the process eliminates the risk of burning the skin. The treatment adheres to the hair, not to the skin, and lubricates the root for easier removal from the hair root.

These factors, combined with the hair removal in the natural direction of growth, create a much more comfortable experience for everyone.

In one embodiment, a skin conditioning program comprises a skin softening and repair step where a natural composition is applied to the skin; a hair extraction and follicle decongestion step where a natural composition is applied to the skin; a follicle detoxification step where a mineral composition is applied to the skin; and skin exfoliation step where a mineral composition is applied to the skin. These program elements are complementary. The skin softening and repair improves the exfoliation; improved exfoliation improves the penetration and efficiency of the follicle detoxification; and the improved detoxification improves paste penetration and sugaring efficiency. The skin conditioning program is designed to establish and to maintain improved skin and hair follicle condition in order to promote more effective hair removal.

### Theory

The Kennedy Theory® for sugaring hair removal using Alexandria Professional® (AP) sugar paste formulations in conjunction with the Kennedy Sugaring Technique Theory™ is as follows:

In one embodiment, the AP sugar paste formulation is applied using the Kennedy Sugaring Technique Application as described in Step 200 below—the “molding process”. This application technique allows the sugar paste to penetrate down into the follicle and to lubricate the hair inside as well as to grip the hair and inner root sheath. Basically, the sugar is sticking to everything on the inside of the follicle that it touches.

Taking a hair in the early anagen stage promotes a faster refinement (thinning) and depletion of the hair, so that the amount of hair and difficulty of removal decrease in subsequent sugaring treatments. When a hair is extracted intact and in its natural direction while in the anagen stage, it has a large bulb traveling through the follicle which takes some of the inner root sheath with it as it is being extracted. The inner root sheath also carries matrix cells responsible for helping to grow the hair. We are all born with a predetermined amount of matrix cells, and by depleting the follicle's inner root sheath time and time again, you help to deplete the hairs growth source.

In addition to the Kennedy Theory®, when you extract a hair in the early anagen stage, you are taking the hair while it is still attached to the derma papilla—its feeding source/nourishment from blood vessels.

Applicant theorizes that extracting a hair in the early anagen stage causes the vessels to be cauterized. Cauterization may also occur instantly from time to time whereby there are no more vessels to continue the nourishment to a given follicle. This explains a reason why sometimes a hair never grows back after a single treatment.

Applicant theorizes that sometimes only some of the vessels become cauterized, and that this partial cauterization is the reason why we see instant refinement results and ongoing refinement results. This phenomena is generally observed if the hair is treated in the early anagen stage which means treatments when the hair is quite short such as ½ mm up to 2 mm.

The length of hair also depends primarily on the location of the hair on the body.

These theories extend the Kennedy Theory®, to explain why the Alexandria Professional® System is successful at providing immediate excellent instant staggered growth and hair refinement—all leading to permanent results.

### BRIEF DESCRIPTION OF DRAWINGS

FIG. 1A is a flow chart illustrating an example skin conditioning program comprising skin softening and repair; skin exfoliation; follicle detoxification; and hair extraction and follicle decongestion.

FIG. 1B is a flow chart illustrating an example skin conditioning program of FIG. 1A where the skin softening and repair step is performed with a PHENOMEN'ALL™ composition; the skin exfoliation step is performed with a SALT SPRING™ composition; the follicle detoxification is performed with a MUD PUDDLE™ composition; and hair extraction and follicle decongestion is performed with a sugaring step.

FIG. 1C is a flow chart illustrating an example skin conditioning program of FIG. 1A where the hair extraction and follicle decongestion is performed with a sugaring step followed by skin soothing with a CC'me™ composition.

FIG. 2 is a flow chart illustrating a sugaring technique for hair removal.

FIG. 3A is a front view illustration of a hand and wrist the “handshake position” of Step 100 of the flow chart of 2.

FIG. 3B is a side view showing the finger and wrist alignment, and finger separation of FIG. 3A.

FIG. 4 is a front view showing the finger and wrist alignment of the sugar paste “molding” of Step 200 of the flow chart of FIG. 2.

FIG. 5 is a front view showing the finger and wrist alignment of the “relax” wrist dropping of Step 300 of the flow chart of FIG. 2.

FIG. 6 is a front view showing the finger and wrist alignment of the “airplane” release of Step 400 of the flow chart of FIG. 2.

FIG. 7A is a front view showing the finger and wrist alignment of the “roll” of Step 500 of the flow chart of FIG. 2.

FIG. 7B is a front view showing the finger and wrist alignment of the “prepare to flick” of Step 500 of the flow chart of FIG. 2.

FIG. 8 is a front view showing the finger and wrist alignment of the “kick off” of Step 600 of the flow chart of FIG. 2.

FIG. 9 is a front view illustration of a hand and wrist the “handshake position” of Step 100 of the flow chart of 2 with sugar paste.

FIG. 10 is a front view showing the finger and wrist alignment and sugar paste of the sugar paste “molding” of Step 200 of the flow chart of FIG. 2.

FIG. 11 is a front view showing the finger and wrist alignment and sugar paste of the “relax” wrist dropping of Step 300 of the flow chart of FIG. 2.

FIG. 12 is a front view showing the finger and wrist alignment and sugar paste of the “airplane” release of Step 400 of the flow chart of FIG. 2.

FIG. 13 is a front view showing the raised hand of the “kick off” of Step 600 of the flow chart of FIG. 2.

FIG. 14 is a front view showing the return to a handshake position following the “kick off” of FIG. 13.

FIG. 15 is a front view showing the molding following the “kick off” and return to a handshake position of FIG. 14.

FIG. 16 is a front view showing the “roll” and return to a handshake position to “prepare to flick” following the “molding” steps of FIG. 14.

FIG. 17 is a front view showing a “kick off” following the “prepare to flick” of FIG. 16.

FIG. 18 is an illustration of a “snap back” following the “kick off” of FIG. 17.

### DESCRIPTION OF EMBODIMENT

### Full Circle of Skin Conditioning™ Program

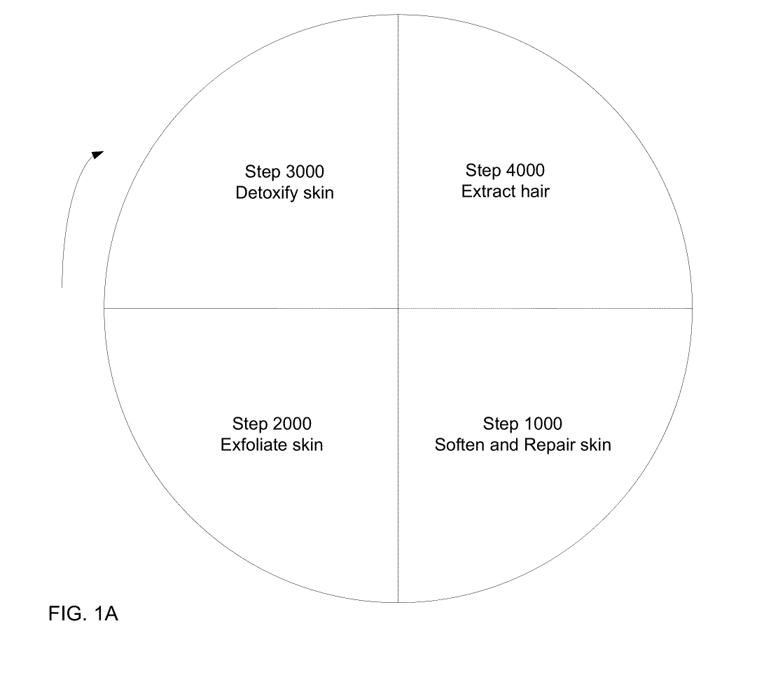

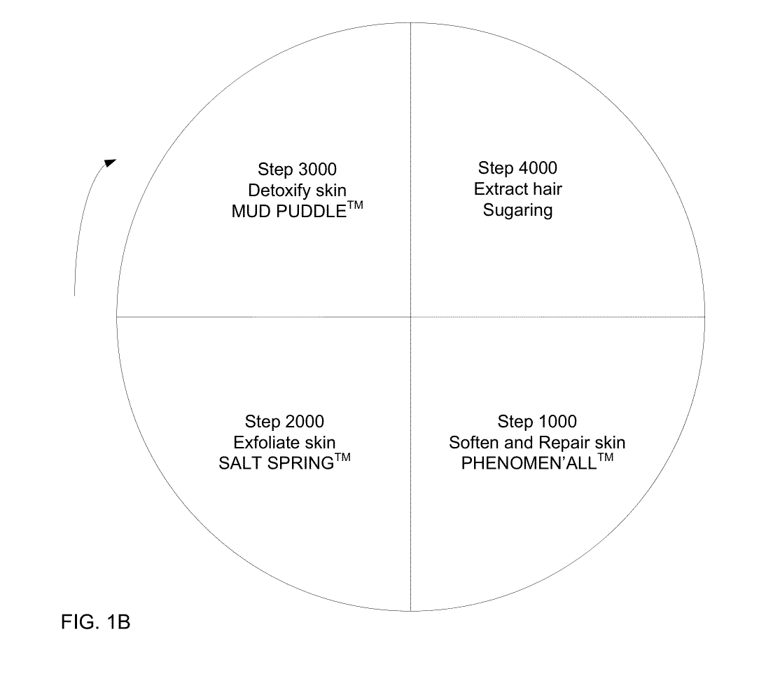

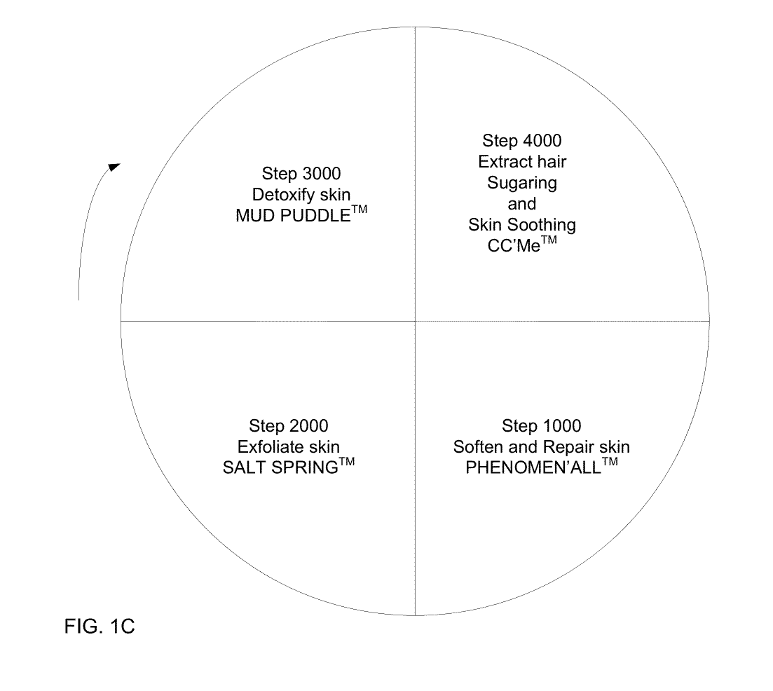

The Alexandria Professional® skin enhancement and improvement, and hair refinement and diminishment program is a proven effective regime used for all skin types and color. FIG. 1 is an illustration of the Full Circle of Skin Conditioning™ Program which comprises skin softening and repair at step 1000; skin exfoliation at step 2000; a follicle detoxification at step 3000; and a hair extraction and follicle decongestion at step 4000.

It takes commitment to the program to correct past problems and to achieve incredible results. Once the skin problems have cleared, applicant recommends a consultation between the aesthetician and the client to establish a maintenance program suited to individual skin and hair needs and to prevent old problems from re-occurring.

The program may be started at any of the steps. No matter which step begins the Full Circle of Skin Conditioning™ Program, the skin color and texture will inevitably improve, and the hair will refine and diminish if the regime recommendations of the program are followed.

### Natural Products and Mineral Products

In this specification, the term “natural product” means a chemical substance which can be produced by a living organism. The substance is considered a natural product even if it can be prepared by total synthesis.

In this specification, the term “composition of natural products” means a composition of one or more natural products.

In this specification, the term “composition of mineral compounds” means a composition of one or more solid substance or composition of natural occurrence, such as Hungarian mud, salts, Manganese Oxide, Phosphorus Pentaoxide, Calcium Carbonate, Magnesium Oxide, Sodium Oxide, Potassium Oxide, and Maris Sal (sea salt).

In this specification, the term “composition of natural products or mineral compounds” means a composition comprising natural products, mineral compounds, or a mixture of natural products and mineral compounds. The composition may include water, but may not include ureas, parabens, or petrochemicals.

### Program Summary

One embodiment of the current invention is a skin conditioning program comprising a skin softening and repair step 1000 comprising the application of a composition of natural products; a hair extraction step 2000 comprising the application of a composition of natural products; a follicle detoxification step 3000 comprising the application of a composition of mineral compounds; and a skin exfoliation step 4000 comprising the application of a composition of mineral compounds. The program may start with any of the cited steps, and one or more steps may be repeated. For instance, the skin softening and repair step may be repeated for several days or weeks before the hair extraction step. The steps may be performed in any order.

FIG. 1B is a flow chart illustrating an example skin conditioning program of FIG. 1A where the steps are performed with compositions of natural products or compositions of mineral products provided by Alexandria Pro. In this example, the skin softening and repair step is performed with a PHENOMEN'ALL™ composition; the skin exfoliation step is performed with a SALT SPRING™ composition; the follicle detoxification is performed with a MUD PUDDLE™ composition; and hair extraction and follicle decongestion is performed with a sugaring step.

### Example

### Phenomen-all® Natural Skin Repair Composition

In one example, the skin softening and repair of step 1000 is accomplished with Phenomen-all® by Alexandria Pro.

Phenomen-all is a skin repair composition which is formulated to repair, refresh and rejuvenate skin. The composition replenishes moisture, calms irritation, and softens skin cells to help relieve ingrown hairs.

It is designed for use everyday by men, women, or children for ingrown hair and skin issues. It can also be used for cuticle treatment and to help smooth out stretch marks and scars. Phenomen-all is an all-natural composition of Butyrospermum Parkii, cocos nucifera, simmondsia chinenis (jojoba) seed oil, sesamum indicum (sesame) seed oil, vitis vinifera (grape) seed oil, aqua, calendula officinalis flower extract, salvia sclarea (clary) oil, aniba rosaeodora (rosewood) wood oil, citrus grandis (grapefruit) peel oil, and cananga odorata flower oil.

Initially, when a problem already exists, such as ingrown hair, bumps, or excess dry skin, applicant recommends the use of Phenomen-all® Skin Repair twice per day—once in the morning and once before bedtime. Phenomen-all® is an at home regimen to treat ingrown hair. Phenomen-all® can be applied to large areas such as legs, or it can be applied to a “spot” area such as an ingrown hair in the bikini line. In either case, the Phenomen-all® should be warmed between the hands, or fingertips for “spots”, and then massaged into the skin. For best penetration results, keep hands in contact with skin, or fingertip contact for “spots”, for 20-30 seconds. The goal is to seep the oil formulation of the Phenomen-all® through the epidermis down to the peripheral layers to help soften and loosen the dead skin cells. This step preps the skin for exfoliation and sloughing.

### Example

### Salt Spring® Natural Skin Repair Composition

After dead skin cells are loosened in the skin softening and repair step, the skin cells are primed for exfoliation. For home use, applicant recommends that the customer mix Salt Spring® from Alexandria Pro, which is a Dead Sea salt, with Restore™ Lotion from Alexandria Pro to slough away all of the softened and loosened skin cells. This is generally a very safe method of exfoliation with no contra-indication concerns as long as there are no skin lesions before beginning the salt glow. Salt glows are considered too abrasive for facial use. Therefore, to benefit from the drawing effects of Dead Sea salt on the face, applicant recommends dissolving 1 teaspoon of Salt Spring® into a small 4-6 ounces bowl of lukewarm water and submerging the chin into the bowl and soaking for 10 minutes. This soaking will help to draw out trapped hair and impurities for those who have issues on the chin and will greatly assist in sugaring treatments. The customer may also submerge disposable gauze towelettes into the water mixture and simply lay the towelettes onto the skin for 10 minutes.

The Salt Spring® skin repair composition is a mineral composition of natural minerals including Magnesium carbonate, potassium hydroxide, sodium chloride, bromide, calcium chloride. The composition rejuvenates the entire body, and leaves skin feeling silky and smooth. This multi-purpose restorative exfoliating treatment helps with ingrown hair problems, treats body blemishes and improves skin texture.

For added aroma splendor, the Alexandria Professional™ Dermawear™ collection formulas can be added to the Salt Spring glow mixture.

### Example

### Mud Puddle™ Follicle Detoxification Composition

After exfoliation, the follicles are more accessible, and applicant recommends Mud Puddle® (Hungarian Wellness Mud®) from Alexandria Pro for follicle detoxification. The Mud Puddle® may be applied as a body wrap to draw out impurities and detoxify the body, skin and follicles. This part of the treatment program is enhanced because the skin has been prepped to allow ease of penetration of the Mud Puddle composition.

Mud Puddle is an all-natural Hungarian mud, which is rich in essential minerals and collagen. This multi-purpose rejuvenating treatment helps with ingrown hair problems, treats facial blemishes and improves skin texture. The composition has long heat retention and is anti-inflammatory. In one example, the composition comprises 60% silicon dioxide which is a building block of collagen from skin to bone, and helps hold collagen tissue together. The composition deeply penetrates tissues and clears stored toxins from the follicle. The composition includes Hungarian Mud, water, sea salt, Lavandula Angustifolia (Lavender Oil), and Commiphora Myrrha (Myrrh).

### Example

### CC-Me™ Skin Smoothing

FIG. 1C is a flow chart illustrating an example skin conditioning program of FIG. 1A where the steps are performed with compositions of natural products or compositions of mineral products provided by Alexandria Pro. In this example, the skin softening and repair step is performed with a PHENOMEN'ALL™ composition; the skin exfoliation step is performed with a SALT SPRING™ composition; the follicle detoxification is performed with a MUD PUDDLE™ composition; and hair extraction and follicle decongestion is performed with a sugaring step followed immediately by [cleaning? and] the application of CC-Me™ skin smoothing product which is a natural composition of Shea butter, soybean oil, Carapa Procera, and Calotropis Procera.

The CC-Me™ skin smoothing reduces potential inflammation. The CC-Me™ skin smoothing is applied to an area where a breakout might be more inclined to occur especially where vellus hair is fully extracted. Vellus hair are shallow hair that sit on the nerve endings, and therefore cause more trauma to the extraction and sometimes causing histamine reactions, etc. The CC-Me™ skin smoothing is applied after making sure the skin is free of all other products including sugar paste so that the penetration is unobstructed and clear to penetrate the skin. It is effective on all skin types and effective upon first application. However, multiple applications may be required with some people in order to get through the “reaction period”.

### Example

### Alexandria Professional® Sugaring Technique

To complete this Circle of Skin Conditioning Program, the sugaring treatments will be easier on both the client and the practitioner because your hair will quickly become less resistant to the extraction and the skin will benefit from the full potential of professional sugaring.

With the Alexandria Professional®sugaring technique, the sugar paste is seeped into the follicles and then removed with a “flicking” action in the natural direction of the hair growth. This action actually extracts hair in the early anagen stage while simultaneously removing debris from the follicles and gently exfoliating the skin as well. Extraction of hair in the early anagen stage, when the hair is generally 1/16″ (less than 1 mm) in length, promotes quicker refinement that leads to diminishment.

Sugaring is an excellent method to refine and diminish hair growth, and simultaneous benefits occur with each treatment. As the sugar paste is molded onto the skin, it naturally seeps into the follicles and sticks to and grips everything it touches inside the follicle including the root of the hair, the debris accumulation from the skin's surface that can become embedded in the follicle, and the inner root sheath carrying matrix cells.

When the quick “flick” action is performed, the sugar paste inside the follicle connected to the sugar paste molded onto the skin is all removed at the same time—taking with it all that is connected to it inside and out. This action is very gentle because the sugar paste acts as a lubricant inside the follicles which allows for a more gentle extraction and allows for the extraction of hair in the natural direction of its growth.

Comodones (blackheads or compacted pores) can also be gently and safely treated with the Alexandria Professional's unique sugaring technique. As described above, the Mud Puddle will help to draw more impurities from the pores in between sugaring treatments.

### Complementary Elements

Each quarter of the Full Circle of Skin Conditioning™ Program plays an intricate part to enhance results from each other. For example, the skin softening and repair improves the exfoliation; improved exfoliation improves the penetration and efficiency of the follicle detoxification; and the improved detoxification improves paste penetration and sugaring efficiency.

A continuous rotation of the Skin Conditioning Program will results in silky smooth skin with tremendous refinement and diminishment of hair.

### Description of Embodiment

### 6 Step Sugaring Technique

The following description is of an improved sugaring technique which can be used in a stand-alone fashion, but which has enhanced results as part of an overall skin conditioning program such as described above. This description is directed at an instructor who is teaching skin care professionals how to use the technique in salons. The technique may also be used at home.

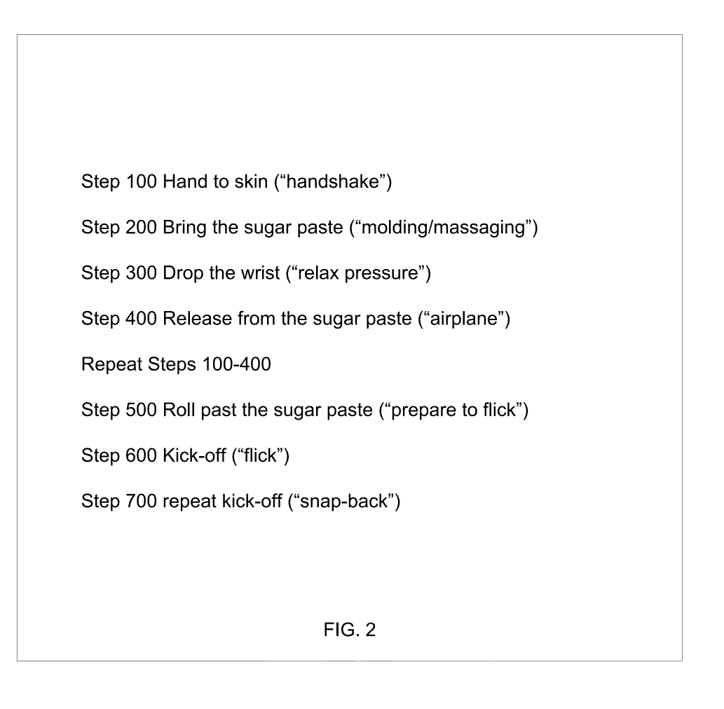

FIG. 2 is a summary of a 6 step sugaring technique used in one embodiment of the current invention.

### Before You Start—Confirming Hand Position

Standing across from each student at the massage table, shake hands with one student at a time and ask them to release from the handshake by dropping the hand to the table. Observe how they place their hand when it reaches the table. Repeat this action until their hand is in the correct handshake position as it reaches the table. Have them observe your hand as it reaches the table so they can mimic it.

This is to establish the correct hand position to begin treatment and the correct hand position to “flick” (remove) the sugar off the skin.

You should never be seeing the top off your hand when in the “hand shake” position.

### Step 100 Hand to Skin (“Handshake Position”)

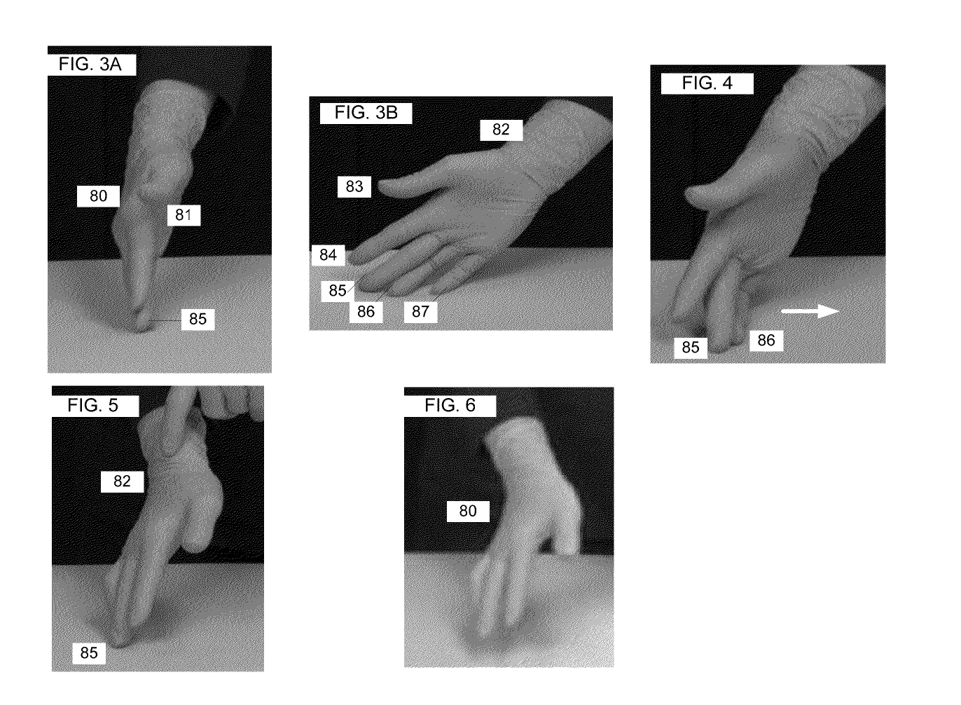

FIG. 3A is a front view illustration of a hand 80 and wrist 82 in a “handshake position” of Step 100 of the flow chart of FIG. 2. FIG. 3B is a side view showing the finger and wrist alignment, and finger separation of FIG. 3A where the middle finger 85, ring finger 86, and baby finger 87 are applying equal pressure. FIGS. 3-8 show hand positions without sugar paste. FIGS. 9-17 show the hand position with sugar paste 92 applied to a portion of a leg 90.

In FIG. 3A, the index finger 84 and thumb 83 are not applying pressure. In FIGS. 9-17, the index finger and thumb are used to help contain the sugar paste.

At step 100, the technician places the sugar paste on the client. The sugar paste is placed on the client's skin with the technician's sugaring hand. The sugar paste is placed on the client's skin, and not in the palm of the technician's hand. The sugar paste is confined to the inside of the technician's fingers and is not present behind the technician's fingers. A controlled and moderate pressure of the technician's fingers 85, 86, and 87 and hand is maintained on the client's skin. In this example, the index finger 84 is not used to apply pressure to the skin. In other examples, the index finger is used to apply equal pressure as the middle finger 85, ring finger 86, and baby finger 87. The thumb 83 of the technician's sugaring hand is used to keep the sugar paste rolling onto the client's skin so that the sugar paste does not move to the technician's palm 81.

FIG. 9 is a front view illustration of a hand 80 and wrist the “handshake position” of Step 100 of the flow chart of 2 with sugar paste 92.

If this step is performed correctly, then there is good penetration and seeping of the sugar paste into the follicles; and the technician is prepared to make an effective and comfortable application of the paste.

### Step 200 Bring the Sugar Paste (“Molding/Massage”)

FIG. 4 is a front view showing the finger and wrist alignment of the sugar paste “molding” of Step 200 of the flow chart of FIG. 2.

FIG. 10 is a front view showing the finger and wrist alignment and sugar paste 92 of the sugar paste “molding” of Step 200 of the flow chart of FIG. 2. The technician's second hand 89 is used to tighten the skin on the leg.

At step 200, the technician maintains a hand position similar to Step 1, with the exception that the technician needs to control the wrist in order to bring the sugar forward. The thumb 83 of the sugaring hand is used to keep the sugar paste 92 off the palm and down on the skin and to keep the sugar paste rolling onto the skin as the technician applies the sugar paste. An even pressure is maintained on all fingertips being used to sugar, and the even pressure helps to keep a solid control of the wrist. The even pressure and wrist control facilitates an even application of the paste. The technician should spread the fingers of the application hand for smoother equal application whilst keeping the fingers in a parallel line to each other. If the fingers are kept too tightly close together, it will affect the application, the time it takes to complete the treatment, especially of legs and arms, and the comfort of the application.

### Step 300 Drop Your Wrist (“Relax/No Pressure”)

FIG. 5 is a front view showing the finger and wrist alignment of the “relax” wrist dropping of Step 300 of the flow chart of FIG. 2.

FIG. 11 is a front view showing the finger and wrist alignment and sugar paste of the “relax” wrist dropping of Step 300 of the flow chart of FIG. 2.

At step 300, the technician immediately stops the application and molding of the sugar paste; and prepares for steps 400, or for steps 500 and 600. As described below, steps 100-400 will typically be repeated once or twice before steps 500 and 600 are executed. If this step is performed incorrectly, then the client may experience discomfort or pain during the release; or may experience discomfort or pain and broken hair when “flicking”.

Step 400 Release from Sugar Paste (“Airplane”)

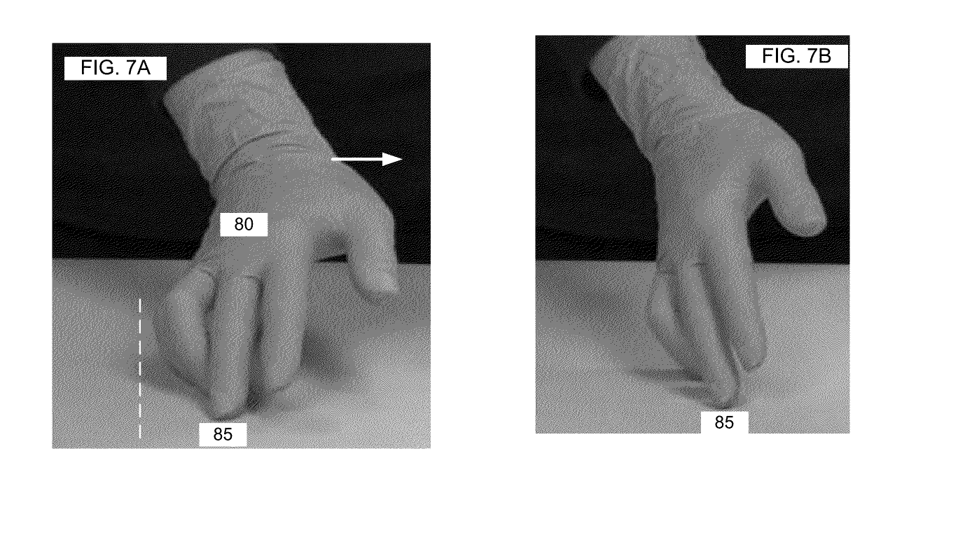

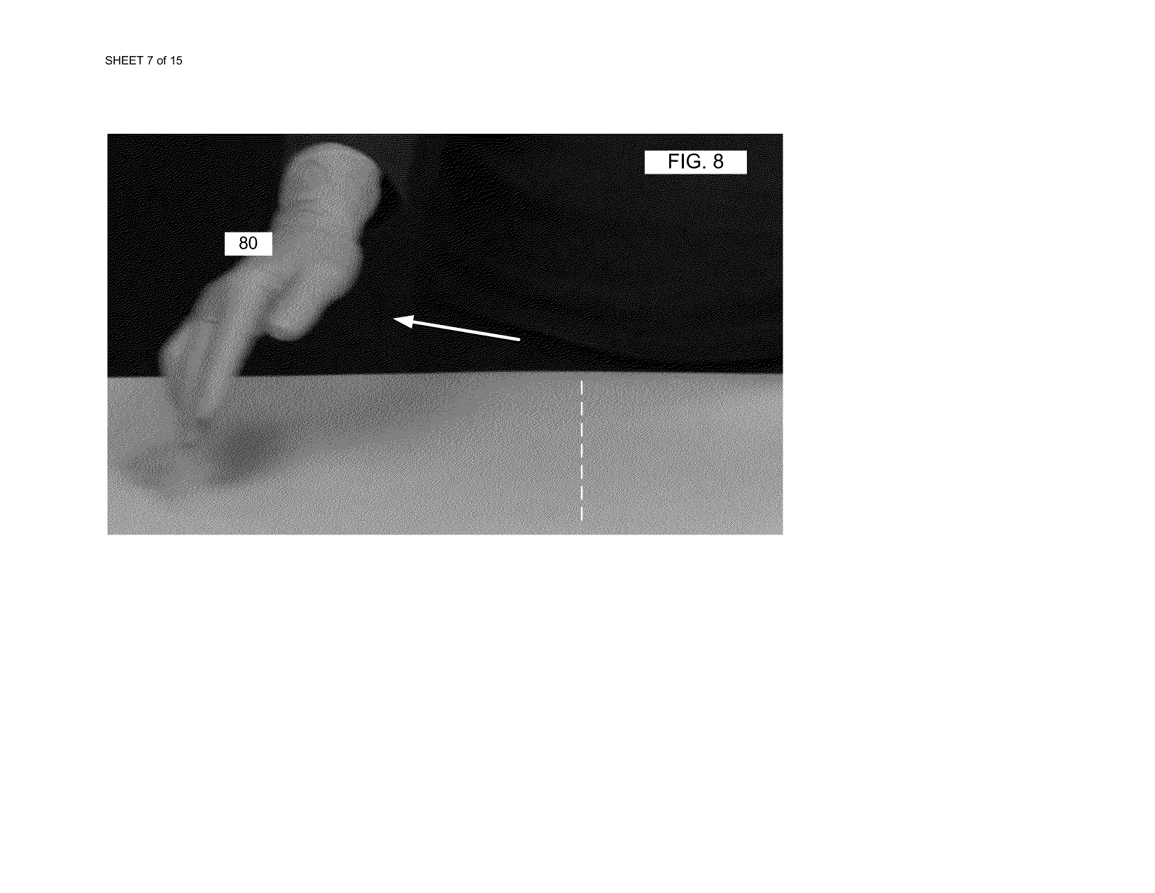

FIG. 6 is a front view showing the finger and wrist alignment of the “airplane” release of Step 400 of the flow chart of FIG. 2.

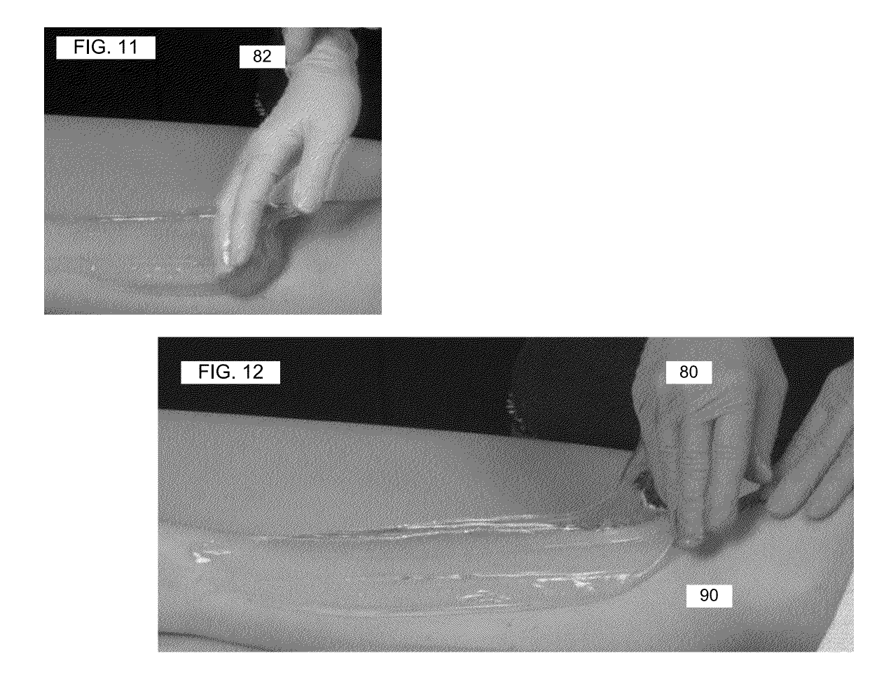

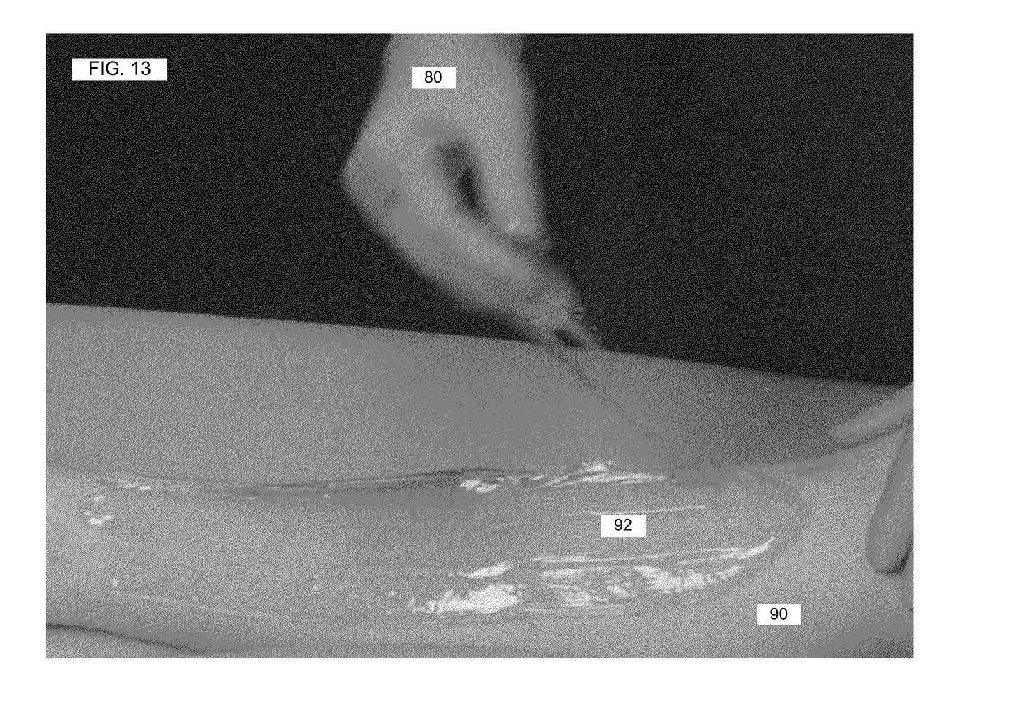

FIG. 12 is a front view showing the finger and wrist alignment and sugar paste of the “airplane” release of Step 400 of the flow chart of FIG. 2.

At step 400, the technician releases the hand from the sugar paste without lifting the skin by executing an “airplane take-off” action—NOT a helicopter. The technician is then properly positioned to repeat steps 100 and 200. If this step is performed incorrectly, then the technician can lift the client's skin or pull the client's hair and cause pain during release. The airplane take-off analogy provides a reminder to the technician to execute a release with the application hand at an acute angle to the skin without abruptly cutting away from the sugar paste.

### Repeat of Steps 100-400

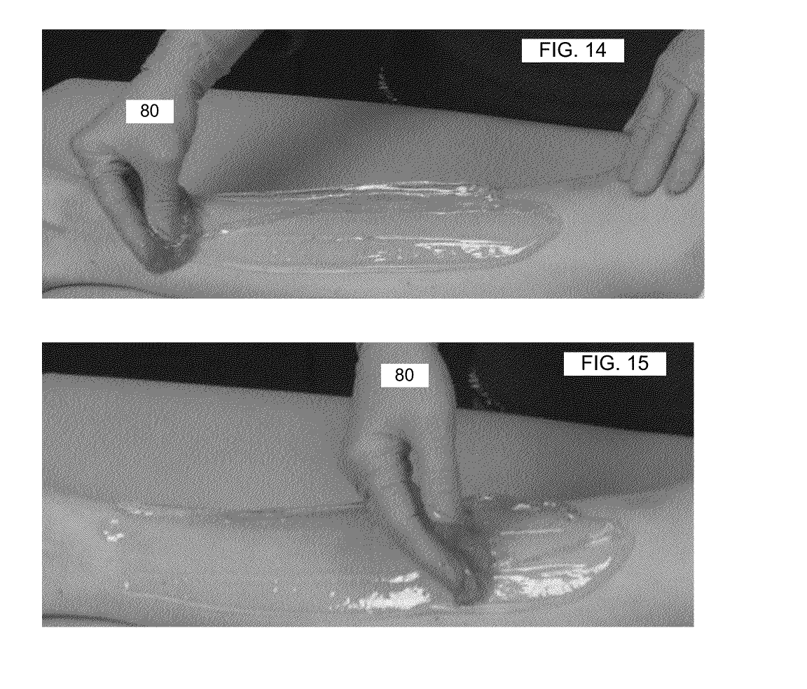

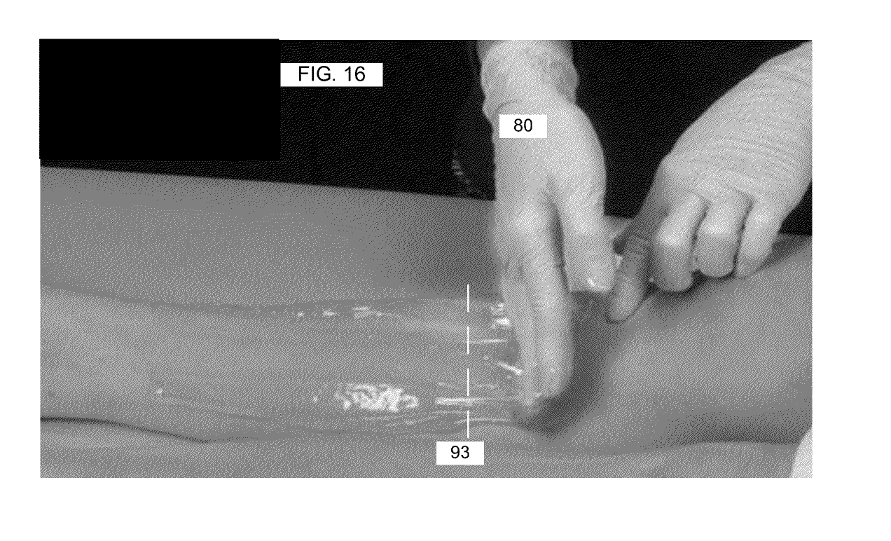

It is generally desirable to repeat the “molding” process once or twice over a region of skin before flicking off the sugar paste. This repeated molding helps to work the sugar into the follicles for better hair removal. FIGS. 14-16 illustrate a repeat of steps 100-200. FIG. 14 is a front view showing the return to a handshake position following the “kick off” of FIG. 13. FIG. 15 is a front view showing the molding following the “kick off” and return to a handshake position of FIG. 14.

### Step 500 Roll Paste Sugar Paste (“Prepare to Flick”)

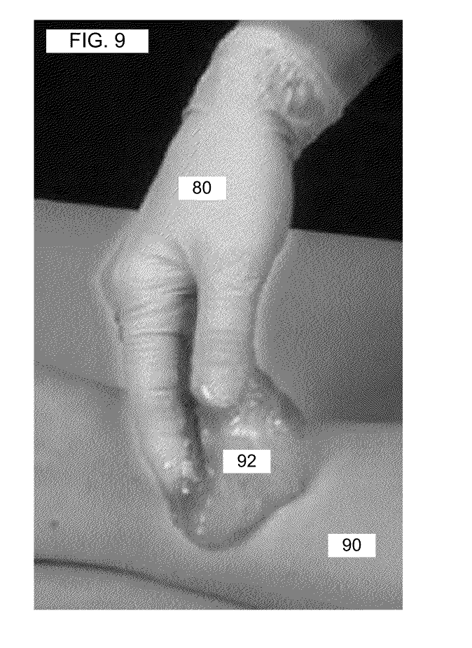

FIG. 7A is a front view showing the finger and wrist alignment of the “roll” of Step 500 of the flow chart of FIG. 2.

FIG. 7B is a front view showing the finger and wrist alignment of the “prepare to flick” of Step 500 of the flow chart of FIG. 2.

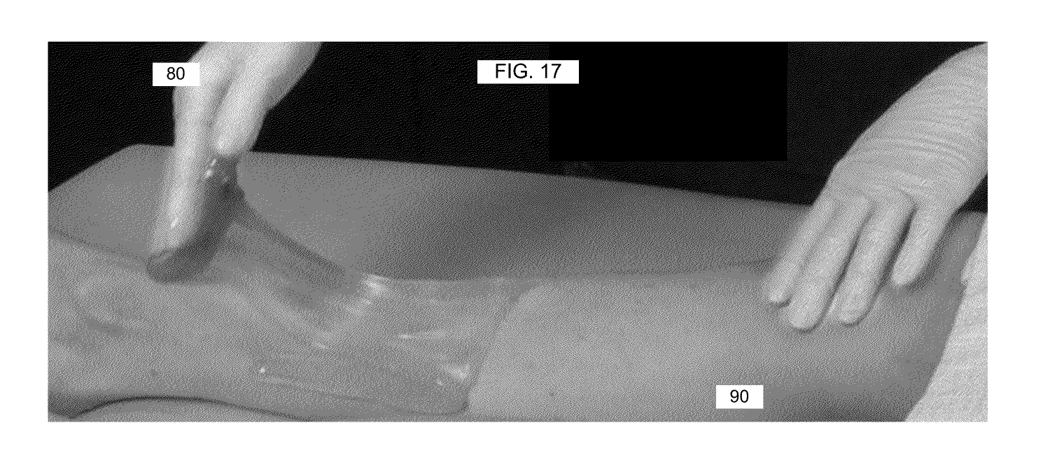

FIG. 16 is a front view showing the “roll” and return to a handshake position to “prepare to flick” following the “molding” steps of FIG. 14.

The sugar paste is not removed during this step. Step 500 should be performed correctly in order to prepare for step 600. The purpose for performing step 500 is to allow the technician to stop with the pressure of step 200 after they have come to step 300, and to “roll past” the applied sugar on the skin while reducing their contact pressure by 50% and continuing to roll on the applied sugar to get to the end of the applied sugar paste. The technician continues to apply equal pressure to all fingers on the application hand. The technician prepares for the “kick-off” (Step 600) by connecting to the end of the sugar paste application. This is similar to removing a piece of tape, by gripping the very edge of the tape to remove it.

### Step 600 Kick-Off (“Flick”)

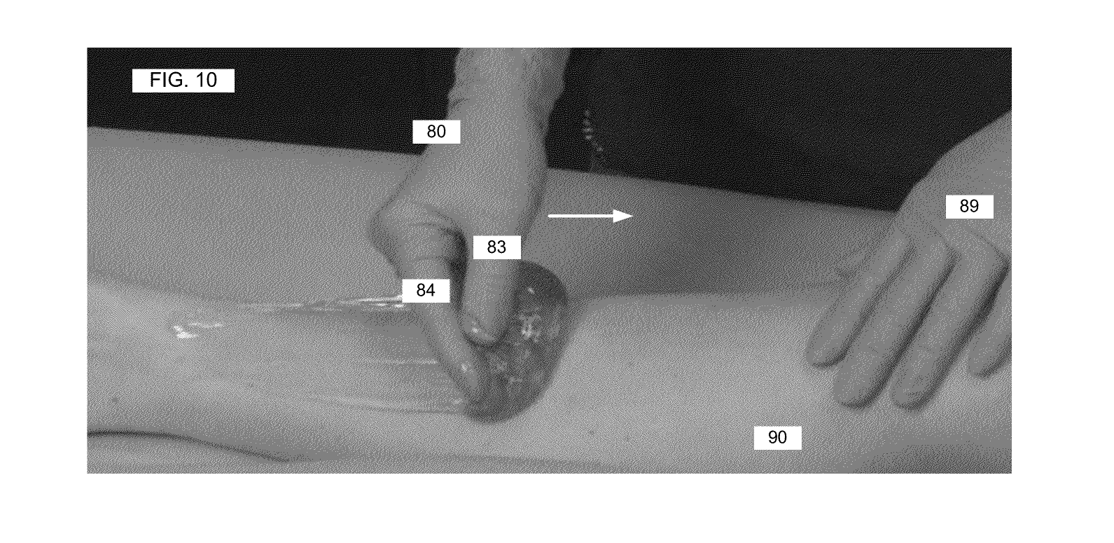

FIG. 8 is a front view showing the finger and wrist alignment of the “kick off” of Step 600 of the flow chart of FIG. 2.

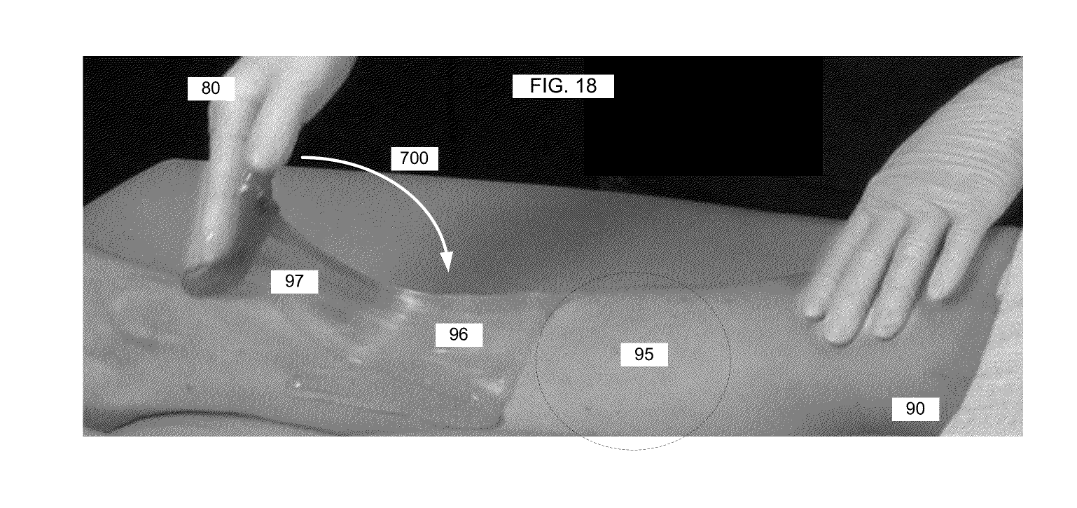

FIG. 17 is a front view showing a “kick off” following the “prepare to flick” of FIG. 16.

At step 600, the technician maintains a comfortable relaxed wrist and application position for the “Kick command” while still moving onto the skin past the applied paste. If the technician sees the palm of the application hand, then the technician should not perform step 600, but should attempt to reposition the hand more into the same relaxed position of step 300. If that fails, the technician should start over at step 100.

The technician's fingers must always remain in a parallel position with equal reduced pressure applied by each finger. A kick-back maneuver is executed without lifting the application hand off the sugar paste. The technician preferably issues a voice command to stress the word “KICK”, and a voice command to stress the word “Handshake” whilst continuing to gently move on the skin and the a final command of the word “OFF”. A firm wrist action is used when performing the “OFF” voice command, with little effort from the technician's shoulder. The “OFF” command should immediately follow the “Handshake” command without the slightest hesitation. If this step is performed incorrectly, then the client may experience discomfort or pain from lifted skin or broken hair. The technician should stay connected to the sugar and skin when “kicking” and when going from a step 300 positioning of the hand.

### Second and Consecutive Kick-Off Using “Snap-Hack” Action (“The Art of Sugaring”)

The technician should immediately snap-back the kicked-off (flicked-off) sugar paste to accumulate it on the client's skin in order to prevent the sugar paste from travelling on the client's skin and to effectively sugar each molded strip while performing professional sugaring treatments. Snap-back will allow the technician to control the sugar paste as it is repeatedly “flicked” off the skin and to prevent it from flying away from the technician's hand and onto the treatment table, the floor and even walls. Snap-back is an intricate part of on-going technique of the 6 Steps to perfect sugaring.

By deciding exactly where one wants to accumulate the “flicked sugar”, the technician can perform step 600 and immediately return her sugaring hand connected to the “flicked sugar paste” to the exact pre-determined spot on the skin. This will allow them to accumulate/gather the sugar paste in a small “pile”, perform step 100 behind the sugar paste pile to collect it and continue to sugar that strip until it is completely off the skin.

FIG. 18 is an illustration of a “snap back” following the “kick off” of FIG. 17. In this figure, the portion 97 of the sugar paste has just been flicked off of area 95 of the leg, and the technician is preparing to “snap back” that portion 97 to area 96 to repeat the “flick” for region 96.

Predetermining where to return the sugaring hand after performing step 600 will help the technician to prevent “travelling on the skin” either in front or behind the applied sugar paste. This will prevent the technician from overworking an area, from getting stuck on the skin and help them to become quicker at the treatment without compromising efficiency.

After each “kick-off” the technician should immediately position the application hand according to step 100 and immediately move to step 300. Step 200 should be eliminated whenever the technician is ready to execute a series of kick off steps. Step 500 (and then step 600) is performed if there is a distance of sugar paste remaining on the skin; and step 600 is performed after step 300 if the sugar paste distance is minimal such as 1 to 2 inches maximum.

If these steps are performed incorrectly, then the client may experience discomfort or pain from lifted skin or broken hair.

Over performing Step 200 will overwork the sugar paste and likely melt it too much into the skin and get the technician stuck. The technician can cause some irritation from trying to Kick-Off sugar paste that is melted into the skin. This can also cause the technician to become too hot from frustration. If this melting occurs, the technician should simply relax, gently remove the application hand from the sugar paste using Step 400, and continue to sugar in another area. When you leave the melted sugar paste for a few minutes, it will cool down and be easier to remove.

### Example

### Series of Sugaring Steps on Client Arm

The technician executes steps 100-600 at a first upper portion of the arm with the application hand.

After the application of the sugar paste on a strip of the upper arm and the initial kick-off, the technician brings the hand to the position of step 100 over a second portion of skin slightly lower on the upper arm, collects the sugar paste that was kicked-off by placing the hand just behind the collected paste, and executes steps 300, step 500 and then step 600. These steps are repeated until the applied strip of sugar paste is completely removed from the skin. (Step 200 is eliminated for several successive kick-off sequences, and step 500 is eliminated when there is less than 2 inches distance of paste remaining on the client's skin.)

After the first applied strip of sugar paste is removed and all the hair beneath the applied sugar paste is extracted, the technician brings the hand to the position of step 100 over another section of the upper arm portion of skin and executes steps 300, 400, 500 and step 600. (Step 200 is eliminated for several successive kick-off sequences, and step 500 is eliminated when there is less than 2 inches distance of paste remaining on the client's skin.)

These steps are repeated until each section of the upper arm is completed and all hair is extracted.

After the upper arm is completed, the technician brings the hand to the position of step 100 over a portion of skin on the underneath part of the lower arm, executes steps 300, 400, 500 and step 600. Afterwards, the same protocol in applying the steps is followed as with the upper arm. Once the underneath portion of the lower arm is completed and all hairs are extracted, the technician then moves to the top forearm. The hair in this portion of the arms grows sideways. The technician follows the natural direction of growth by positioning the hand to step 100 and executes step 200, 300, 400, 500 and step 600 respectively from the outside of the forearm to the inside of the forearm.

After the final kick-off of the first strip applied to the lower top forearm, the technician brings the hand to the position of step 100 over a new portion of the skin on the forearm, and executes steps 100-600.

The technician must always inspect the direction of the hair growth prior to beginning treatments using the 6 Steps. The Kennedy Theory® executes the extraction of hair only in the natural direction of hair growth, and this requires that Steps 100 and 200 be executed against the natural direction for better penetration of the sugar paste and an even gentler execution of step 600.

Each Step has magic to its performance. And, each Step relies on each other for perfect technique performance. When the technician fully understands the relevance of immediate sequencing for some of these Steps, the technician will be able to learn how to effectively extract hair less than 1 mm by applying T.H.E. Technique™ when it is called upon. (Tough Hair Extraction Technique)

### SUMMARY

With some patience the technician can gain experience and to master the magic of Alexandria Professional™ body sugaring hair removal and skin conditioning treatments.

Step 100 provides the technician full control of sugar paste, establishes her positioning onto the skin and the correct pressure.

Step 200 allows the technician to mold/apply the sugar paste without causing pain or discomfort to the client and to seep the sugar paste into the follicle.

Step 300 allows the technician to prepare for either the release of the hand from the sugar and skin, or the removal of the sugar paste without causing pain or discomfort to the client.

Step 400 allows the technician to return to continual Step 2 molding process without causing pain or discomfort.

Step 500 allows the technician to prepare for the removal of the sugar paste without causing pain or discomfort.

Step 600 allows the technician to effectively remove the sugar paste and extract the hair and to perform the “snap-back” without causing pain or discomfort. After each “kick-off” the technician should immediately position the application hand according to step 100 and immediately move to step 300.

### Example

### Permissible Variations

When you are ready to prepare for the Kick-off, you no longer need to perform step 200. If you continue with step 200 every time you perform step 600, then you will overwork the sugar paste and the skin and cause the sugar to melt too much into the skin.

The purpose of step 400 is to correctly release from the sugar paste on the skin without causing discomfort when you are going back to repeat the molding step 200.

You only need to use step 500 when there is enough sugar paste molded onto the skin for you to “roll past” in preparation for the Kick-Off If there is only a small piece of sugar paste such as the very last Kick-Off, then you do not use step 500, but perform steps 100, 300, and 600.

### Example

### Non-Permissible Variations in Preferred Embodiment

In the preferred embodiment, the sugaring technique is disciplined and several variations are non-permissible.

Every time that you return to/on the skin, you should be in the handshake position of step 100, even if you are “cleaning up” the skin.

Every time that you are ready to either release from the sugar paste, or to get ready to remove the sugar paste, you must perform step 300.

Every time you are taking sugar paste OFF the skin, you must use step 600, and be in a handshake position. This includes Kick-Offs and clean-ups.

While exemplary embodiments of the invention have been described, it should be apparent that modifications and variations thereto are possible, all of which fall within the true spirit and scope of the invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. A skin conditioning program comprising
a skin softening and repair step comprising
the application of a composition of natural products;
a skin exfoliation step comprising
the application of a composition of mineral compounds;
a follicle detoxification step comprising
the application of a composition of mineral compounds; and
a hair extraction step comprising
the application of a sugar composition of natural products, such that the program may start with any of the cited steps.

2. The skin conditioning program of claim 1 wherein the skin softening and repair step further comprises
the application of a composition of Butyrospermum Parkii, cocos nucifera, simmondsia chinenis (jojoba) seed oil, sesamum indicum (sesame) seed oil, vitis vinifera (grape) seed oil, aqua, calendula officinalis flower extract, salvia sclarea (clary) oil, aniba rosaeodora (rosewood) wood oil, citrus grandis (grapefruit) peel oil, and cananga odorata flower oil.

3. The skin conditioning program of claim 1 wherein the skin exfoliation step further comprises
the application of a composition of natural minerals including Magnesium carbonate, potassium hydroxide, sodium chloride, bromide, calcium chloride.

4. The skin conditioning program of claim 1 wherein the skin exfoliation step further comprises
the application of a composition of Hungarian Mud, water, sea salt, Lavandula Angustifolia (Lavender Oil), and Commiphora Myrrha (Myrrh).

5. The skin conditioning program of claim 1 wherein the hair extraction step further comprises
the application of a sugaring composition comprising sugar, water, and lemon oil.

6. The skin conditioning program of claim 5 wherein the hair extraction step further comprises
cleaning the skin after the application of the sugaring composition, and
applying a skin soothing composition of natural products comprising Shea butter, soybean oil, Carapa Procera, and Calotropis Procera.

7. The skin conditioning program of claim 1 wherein the hair extraction step further comprises
placing a quantity of a sugaring composition at a beginning position on a strip of skin;
spreading, by hand, the quantity of the sugaring composition to form a deposited layer of sugaring composition over the strip of skin, such that the deposited layer extends from the beginning position to a leading edge;
flicking off a lead portion of sugaring composition from the deposited layer of sugaring composition in proximity to the leading edge of the layer, such that hair is removed from the skin by the lead portion of the deposited layer of the sugaring composition, thereby leaving a remaining portion and remaining lead edge of the deposited layer on the skin;
snapping back the lead portion of the sugaring composition to the remaining lead edge; and
flicking off a portion of the remaining layer of the sugaring composition in proximity to the remaining lead edge.

8. The skin conditioning program of claim 7 further comprising
continuing the steps of snapping back and flicking off remaining edge portions of the layer of sugaring composition until the sugaring composition has been removed from the beginning position.

9. The skin conditioning program of claim 7 wherein spreading, by hand, the lump of the sugar composition to form a deposited strip of sugar composition over the region of skin further comprises
returning to the beginning position and repeating the spread of sugaring compostion over the strip of skin.

10. A method of teaching a skin conditioning program, the method comprising
teaching the manual application of a sugar composition to extract hair by demonstrating a sequence of hand, wrist, and finger positions, the sequence comprising
an initial handshake position to begin the deposition of the sugar composition to a region of skin with an initial deposition pressure,
a molding stroke to apply, with the initial deposition pressure, a layer of the sugar composition over the region of skin,
a relax wrist pressure position to reduce the pressure of the molding stroke,
a release position to stop the deposition of the sugar composition,
a prepare to flick position,
a rapid flick stroke to remove a first portion of the sugar composition from the skin, and
a snap back stroke to position the hand to execute a subsequent flick stroke to remove a first portion of the sugar composition from the skin.

11. The method of claim 10 further comprising
teaching the application of a composition of natural products to soften and repair skin.

12. The method of claim 10 further comprising
teaching the application of a composition of mineral compounds to exfoliate skin.

13. The method of claim 10 further comprising
teaching the application of a composition of mineral compounds to detoxify skin follicles.
